(19) United States
(12) Patent Application Publication
(10) Pub. No.: US 2024/0343570 A1
(43) Pub. Date: Oct. 17, 2024

(54) REACTIVE OXYGEN SUPPLY APPARATUS, TREATMENT APPARATUS USING REACTIVE OXYGEN, AND TREATMENT METHOD USING REACTIVE OXYGEN

(71) Applicant: CANON KABUSHIKI KAISHA, Tokyo (JP)

(72) Inventors: MOTOTERU GOTO, Shizuoka (JP); KAZUHIRO YAMAUCHI, Shizuoka (JP); TAKUMI FURUKAWA, Shizuoka (JP); MASAKI OZAWA, Shizuoka (JP); KENJI TAKASHIMA, Kanagawa (JP); SHOTA KANEKO, Tokyo (JP)

(21) Appl. No.: 18/751,446

(22) Filed: Jun. 24, 2024

Related U.S. Application Data
(63) Continuation of application No. PCT/JP2022/048034, filed on Dec. 26, 2022.

(30) Foreign Application Priority Data
Dec. 28, 2021 (JP) 2021-215340

(51) Int. Cl. C01B 13/02; A61L 2/20; H05H 1/24

(52) U.S. Cl. CPC C01B 13/0203; A61L 2/20; H05H 1/2418; H05H 1/2425; A61L 2202/11; H05H 2245/36; H05H 2245/40

(57) ABSTRACT

This reactive oxygen supply apparatus is characterized by comprising a humidification device, a housing having at least one opening, and a predetermined plasma actuator in the opening, and is characterized in that the humidification device humidifies the inside of the housing and generates reactive oxygen in the induced flow, so that the induced flow contains the reactive oxygen, and the plasma actuator and the humidification device are disposed such that the induced flow containing the reactive oxygen flows out of the housing through the opening.

### CROSS-REFERENCE TO RELATED APPLICATION

This is a continuation of International Application No. PCT/JP2022/048034, filed on Dec. 26, 2022, and designated the U.S., and claims priority from Japanese Patent Application No. 2021-215340 filed on Dec. 28, 2021, the entire contents of which are incorporated herein by reference.

### BACKGROUND OF THE INVENTION

### Field of the Invention

The present disclosure relates to an active oxygen supply device, a treatment device using active oxygen, and a treatment method using active oxygen.

### Description of the Related Art

In recent years, ozone generators have become pervasive for indoor or in-vehicle air cleaning, refrigerator deodorizing, nosocomial sterilization, and the like. This is because ozone is oxidative. Further, ozone is decomposed when humidified. At this point, the ozone first becomes more strongly oxidizing active oxygen and is later decomposed into oxygen.

Japanese Patent Application Laid-open No. H10-328279 discloses a method in which the inside of a casing is humidified in advance by a unit that humidifies the inside of the casing to bring an object to be treated into a wet state, and then the object in the wet state is caused to come into contact with ozone, thereby obtaining a high sterilization operation due to active oxygen.

### SUMMARY OF THE INVENTION

After studies by present inventors about sterilization performance obtained by the sterilization method according to Japanese Patent Application Laid-open No. H10-328279, the sterilization performance becomes approximately equal to sterilization performance obtained by a conventional sterilization method using only ozone in some cases. The study result is beyond their expectation despite that the sterilization performance of active oxygen is believed to significantly surpass that of ozone.

At least an aspect of the present disclosure is aimed at providing an active oxygen supply device capable of more efficiently supplying active oxygen to the surface of an object to be treated.

Further, at least an aspect of the present disclosure is aimed at providing a treatment device using active oxygen capable of more efficiently treating the surface of an object to be treated using active oxygen.

Moreover, at least an aspect of the present disclosure is aimed at providing a treatment method using active oxygen capable of more efficiently treating the surface of an object to be treated using active oxygen.

At least one aspect of the present disclosure provides an active oxygen supply device comprising:
a humidifying device;
a housing having at least one opening part; and
a plasma actuator arranged inside of the housing, wherein
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the humidifying device humidifies the inside of the housing to generate active oxygen in the induced flow, and the induced flow results in an induced flow containing the active oxygen, and
the plasma actuator and the humidifying device are arranged so that the induced flow containing the active oxygen flows to an outside of the housing from the opening part.

Further, at least one aspect of the present disclosure provides a treatment device treating a surface of an object to be treated using active oxygen, the treatment device comprising:
a humidifying device;
a housing having at least one opening part; and
a plasma actuator arranged inside of the housing, wherein
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the humidifying device humidifies the induced flow containing the ozone to generate active oxygen in the induced flow, and the induced flow results in an induced flow containing the active oxygen, and
the plasma actuator and the humidifying device are arranged so that the induced flow containing the active oxygen flows to an outside of the housing from the opening part.

Furthermore, at least one aspect of the present disclosure provides a treatment method for treating a surface of an object to be treated using active oxygen, the treatment method comprising:
a step of preparing a treatment device using active oxygen, wherein
the treatment device using the active oxygen comprises
   a humidifying device,
   a housing having at least one opening part, and
   a plasma actuator arranged inside of the housing,
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the humidifying device humidifies the induced flow containing the ozone to generate active oxygen in the induced flow, and the induced flow results in an induced flow containing the active oxygen, and
the plasma actuator and the humidifying device are arranged so that the induced flow containing the active oxygen flows to an outside of the housing from the opening part,
the treatment method further comprising
a step of arranging the prepared treatment device using the active oxygen and the object to be treated at relative positions so that the surface of the object to be treated is exposed when the induced flow containing the active oxygen flows out from the opening part, and
a step of causing the induced flow to flow out from the opening part to treat the surface of the object using the active oxygen.

At least an aspect of the present disclosure enables the provision of an active oxygen supply device capable of more efficiently supplying active oxygen to the surface of an object to be treated. Further, at least an aspect of the present disclosure enables the provision of a treatment device using active oxygen capable of more efficiently treating the surface of an object to be treated using active oxygen. Moreover, at least an aspect of the present disclosure enables the provision of a treatment method using active oxygen capable of more efficiently treating the surface of an object to be treated using active oxygen. Further features of the present invention will become apparent from the following description of exemplary embodiments with reference to the attached drawings.

### BRIEF DESCRIPTION OF THE DRAWINGS

FIGS. 1A and 1B are schematic cross-sectional views showing the configuration of an active oxygen supply device according to an aspect of the present disclosure;

FIGS. 2A and 2B are schematic cross-sectional views showing the configuration of a plasma actuator according to an aspect of the present disclosure;

FIG. 3 is an explanatory view of the plasma actuator according to an aspect of the present disclosure;

FIGS. 4A and 4B are schematic views showing the relationship between a first electrode and a second electrode; and

FIGS. 5A to 5C are schematic views of doughnut-shaped electrodes.

### DESCRIPTION OF THE EMBODIMENTS

Hereinafter, embodiments for carrying out the present disclosure will be specifically illustrated with reference to the drawings. However, the dimensions, materials, shapes, their relative arrangements, or the like of constituting components described in the embodiments shall be appropriately modified depending on the configurations or various conditions of members to which the disclosure is applied. That is, the range of the disclosure does not intend to be limited to the following embodiments.

In the present disclosure, statements of “from XX to YY” and “XX to YY” each representing a numerical value range mean numerical value ranges including lower limits and upper limits, which are endpoints, unless otherwise particularly specified. When numerical value ranges are stepwise stated, the upper and lower limits of the individual numerical value ranges can optionally be combined. In addition, in the present disclosure, such a statement as, e.g., “at least one selected from the group consisting of XX, YY, and ZZ” means any of XX, YY, ZZ, a combination of XX and YY, a combination of XX and ZZ, a combination of YY and ZZ, and a combination of XX, YY, and ZZ.

Further, “funguses” as targets for “sterilization” according to the present disclosure represent microorganisms, and the microorganisms include, in addition to true funguses, bacteria, single-celled algae, viruses, protozoans, or the like, animal or plant cells (including stem cells, dedifferentiated cells, and differentiated cells), tissue cultures, fused cells (including hybridoma) obtained by gene engineering, dedifferentiated cells, and transformants (microorganisms). Examples of viruses include noroviruses, rotaviruses, influenza viruses, adenoviruses, coronaviruses, measles viruses, rubella viruses, hepatitis viruses, herpes viruses, HIV, or the like. Further, examples of bacteria include Staphylococcus bacteria, E. coli bacteria, Salmonella, Pseudomonas aeruginosa, cholera bacteria, dysentery bacilli, Bacillus anthracis, tubercle bacilli, botulinum, tetanus bacilli, Streptococcus, or the like. Moreover, examples of true funguses include Trichophyton, Aspergillus, Candida, or the like. Accordingly, the “sterilization” according to the present disclosure includes, for example, the deactivation of viruses.

Moreover, active oxygen in the present disclosure includes, for example, free radicals such as superoxide (·O₂⁻) and hydroxy radicals (·OH) produced by the decomposition of ozone (O₃).

Moreover, configurations having the same functions will be denoted by the same symbols in the drawings, and their descriptions will be omitted depending on circumstances below.

Furthermore, in the present specification, an active oxygen supply device according to the present disclosure and a treatment device using active oxygen according to the present disclosure will also be generically and simply called an “active oxygen supply device.”

According to studies by the present inventors, it is presumed that one reason for restrictive sterilization performance obtained by a sterilization device according to Japanese Patent Application Laid-open No. H10-328279 is as follows.

According to Japanese Patent Application Laid-open No. H10-328279, the inside of a casing is humidified by a unit that humidifies the inside of the casing to bring an object to be treated into a wet state. Subsequently, the object in the wet state is caused to come into contact with ozone, thereby exciting the ozone and generating highly oxidative active oxygen. Here, the active oxygen refers to a generic term of highly reactive active oxygen species such as superoxide anion radical (·O₂⁻) and hydroxyl radical (·OH), and is capable of immediately oxidizing and decomposing bacteria or viruses with its own high reactivity.

However, in the sterilization device according to Japanese Patent Application Laid-open No. H10-328279, it is presumed that active oxygen is generated only near an ozone supply unit when the humidifying unit and the ozone supply unit are operated at the same time. That is, it is presumed that moisture does not sufficiently reach ozone present at a position distant from the ozone supply unit, thus making it difficult for active oxygen to be generated at a place distant from the ozone supply unit.

Further, it is presumed that active oxygen is highly unstable, extremely short with a half-life of 10⁻⁶ seconds for ·O₂⁻ and a half-life of 10⁻⁹ seconds for ·OH, and promptly converted into stable oxygen and water. Therefore, it is presumed that active oxygen generated near the ozone supply unit is hardly supplied to an object to be treated such as a sterilization target. In other words, it is presumed that, when the humidifying unit and the ozone supply unit are operated at the same time in the positional relationship as illustrated in Japanese Patent Application Laid-open No. H10-328279, the sterilization of a sterilization target at a position distant by at least 1 cm from the ozone supply unit is substantially achieved by ozone.

Therefore, it is presumed that sterilization performance obtained by a sterilization method according to Japanese Patent Application Laid-open No. H10-328279 becomes approximately equal to sterilization performance obtained by a conventional sterilization method using only ozone.

By such considerations, the present inventors have recognized that it is necessary to more actively place an object to be treated or a surface to be treated under an atmosphere of active oxygen in order to treat the object using the active oxygen. After studies based on this recognition, the present inventors have found that the active oxygen supply device as will be described below is capable of more actively placing an object to be treated under an atmosphere of active oxygen. Note that in the present disclosure, the “treatment” of an object to be treated using active oxygen includes any treatment achievable by active oxygen, such as surface modification (hydrophilic treatment), sterilization, deodorization, and bleaching of the surface to be treated of the object using active oxygen.

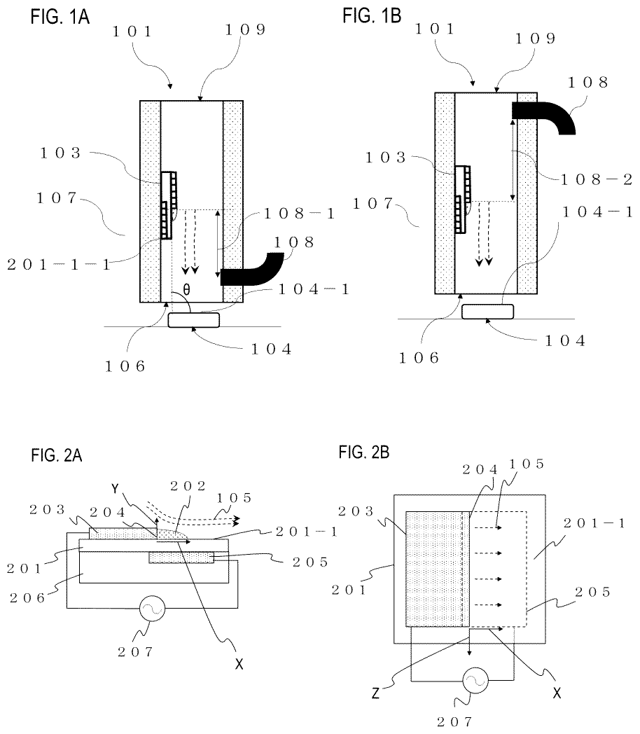

Hereinafter, an active oxygen supply device (a treatment device using active oxygen) 101 according to an aspect of the present disclosure will be described using FIGS. 1A and 1B. The active oxygen supply device 101 according to the aspect of the present disclosure comprises: a humidifying device (not shown); a housing 107 having at least one opening part 106, and a plasma generation device (plasma actuator) 103 arranged inside of the housing.

The humidifying device is arranged to be capable of humidifying the inside of the housing, and generates active oxygen in an induced flow 105. For example, in FIG. 1A, the tip end of an emission unit 108, which is connected to the humidifying device and emits moisture from the humidifying device, is arranged inside of the housing, thus enabling the humidification of the inside of the housing with the humidifying device. In FIG. 1A, symbol 104 denotes an object to be treated.

An aspect of the cross-sectional structure of the plasma actuator 103 is shown in FIG. 2A. The plasma actuator is a so-called dielectric barrier discharge (DBD) plasma actuator (that will be simply called “DBD-PA” below depending on circumstances) in which a first electrode 203 with its end surface exposed is provided on one surface (that will also be called a “first surface” below) of the dielectric 201, and in which a second electrode 205 is provided on the surface (that will also be called a “second surface” below) on the side opposite to the first surface. In FIG. 2A, sign 206 denotes a dielectric substrate to embed the second electrode 205 in the plasma actuator in the thickness direction to prevent the generation of an induced flow from the end surface of the second electrode. Further, the application of a voltage to both the first electrode and the second electrode is possible with a power supply 207. In the plasma actuator 103, the first electrode 203 and the second electrode 205, which are arranged via the dielectric 201, are arranged diagonally opposite to each other. By the application of a voltage between these electrodes (both the electrodes) from the power supply 207, a dielectric barrier discharge oriented from the first electrode 203 toward the second electrode 205 is generated. Then, a jet-like flow is induced by plasma 202 along an exposed part (a part not coated with the first electrode) 201-1 of the first surface of the dielectric 201 from an edge part 204 of the first electrode 203 toward the extending direction (an arrow X direction in FIG. 2A) of the second electrode. At the same time, an air-sucking flow oriented from the internal space of a container toward to the electrodes is also generated. Electrons in the surface plasma 202 collide with oxygen molecules in the air, and dissociate the oxygen molecules to generate oxygen atoms. After the generated oxygen atoms collide with undissociated oxygen molecules, ozone is generated. Accordingly, an induced flow 105 containing the concentrated ozone is generated along the surface of the dielectric 201 from the edge part 204 of the first electrode 203 by the operation of the jet-like flow by the surface plasma 202 and the air-sucking flow.

Further, the plasma actuator 103 and the humidifying device are arranged so that the induced flow 105 containing active oxygen flows to the outside of the housing 107 from the opening part 106 and is supplied to a treatment surface 104-1 of the object 104 to be treated.

That is, the plasma actuator comprises the first electrode 203, the dielectric 201, and the second electrode 205 laminated together in this order, and the first electrode 203 is an exposed electrode provided on the first surface representing one surface of the dielectric 201. When a voltage is applied between the first electrode 203 and the second electrode 205, the plasma actuator generates a dielectric barrier discharge oriented from the first electrode 203 toward the second electrode 205, and blows out an induced flow toward the first direction representing one direction along the surface of the dielectric 201 from the first electrode 203.

More specifically, the plasma actuator generates a dielectric barrier discharge oriented from the edge part 204 on one side of the first electrode 203 toward the second electrode 205, and blows out an induced flow representing a unidirectional jet flow in the first direction (a direction indicated by arrows 105 in FIG. 2A) along the first surface of the dielectric 201 from the edge part 204 on the one side of the first electrode 203.

Further, the second electrode 205 extends in the blowing-out direction (first direction) of an induced flow in one cross section of the plasma actuator in the thickness direction.

More specifically, for example, the plasma actuator comprises the dielectric 201. When the cross section of the plasma actuator in the thickness direction is viewed, the first electrode 203 and the second electrode 205 are arranged diagonally opposite to each other via the dielectric 201 in the thickness direction of the plasma actuator. The first electrode 203 is provided to coat a part of the first surface of the dielectric 201, and the first surface of the dielectric has the exposed part 201-1 not coated with the first electrode 203.

FIG. 2B is a view of the plasma actuator when viewed through from the side of the first surface of the dielectric. At least a part of the exposed part 201-1 and the second electrode 205 indicated by a broken line overlap each other. Accordingly, the overlap represents the region formed by the upper, lower, and right sides of the broken line indicating the electrode 205 and the edge part 204 in FIG. 2B.

By the application of a voltage between the first electrode and the second electrode, an induced flow containing ozone is generated along the exposed part of the dielectric overlapping the second electrode 205 in the cross section (FIG. 2A) in the thickness direction, originating from the edge part 204 of the first electrode 203 on the side of the first direction.

Note that in the present disclosure, the first direction along the surface of the dielectric representing the blowing-out direction of an induced flow as shown in FIGS. 2A and 2B will be defined as an X-direction depending on circumstances. Further, an axis including the X-direction will be defined as an X-axis. For example, an X-axis direction collectively includes the X-direction and the direction opposite to the X-direction. The axis perpendicular to the X-axis and perpendicular to the first surface of the dielectric will be defined as a Y-axis. In the Y-axis, the direction of the first electrode when viewed from the dielectric (for example, a substantially vertically upward direction when the dielectric is set in a horizontal position) will be defined as a Y-direction. The direction perpendicular to the X-direction and along the first surface of the dielectric, that is, an axis perpendicular to the X-axis and Y-axis will be defined as a Z-axis.

An induced flow transforms into, for example, a wall-surface jet flow along the exposed part 201-1, and easily supplies concentrated ozone to a specific position. The length of the exposed part 201-1 in the direction of an induced flow (that is, the length from the edge part 204 of the first electrode on the side of the first direction to the edge part of the first surface of the dielectric) is not particularly limited but is preferably in the range from 0.1 mm to 50 mm, more preferably in the range from 0.5 mm to 20 mm, and still more preferably in the range from 1.0 mm to 10 mm.

In the active oxygen supply device according to an aspect of the present disclosure, the induced flow 105 containing ozone from the plasma actuator 103 flows to the outside of the housing 107 from the opening part 106, and is supplied to the treatment surface 104-1 of the object 104 to be treated. At the same time, the humidifying device humidifies the induced flow 105 to generate active oxygen in the induced flow 105 and the induced flow results in an induced flow containing the active oxygen. Thus, it is possible to actively supply the active oxygen to a region near the treatment surface 104-1, specifically, a spatial region (that will also be called a “surface region”) approximately 1 mm above the treatment surface. Therefore, it is possible to supply the active oxygen to the surface of the object to be treated before the generated active oxygen is converted into oxygen and water. As a result, the treatment surface 104-1 of the object 104 is reliably treated by the active oxygen.

Further, concentrated ozone is contained in the induced flow 105, thus eliminating the need to increase ozone concentration in the space between the humidifying device and the surface region. This makes it possible to prevent moisture from the humidifying device from decreasing before reaching the surface region. As a result, the ozone present in the surface region is efficiently decomposed into active oxygen by the moisture. Then, the active oxygen is generated on the treatment surface 104-1 of the object to be treated or at a position extremely close to the treatment surface 104-1. Then, the treatment surface 104-1 of the object 104 to be treated is placed under an atmosphere of the active oxygen generated in situ on the treatment surface, and the treatment surface is reliably sterilized by the active oxygen.

Further, the induced flow 105 is generated by the plasma actuator 103, thus eliminating the need to install an airflow generation device such as a blowing fan. As a result, there is no hinderance to ozone generation caused by an airflow from a fan or the like or no diffusion of generated ozone. Therefore, it is possible to efficiently supply the active oxygen to the treatment surface 104-1. That is, the active oxygen supply device preferably does not include an airflow generation device such as a blowing fan that generates an airflow to supply the induced flow 105 to the treatment surface 104-1. The induced flow 105 itself generated from the plasma actuator 103 is preferably supplied to the treatment surface 104-1 of the object 104 to be treated. The induced flow 105 supplied to the treatment surface 104-1 is, for example, a jet-like flow.

### Electrodes and Dielectric

Materials constituting the first electrode and the second electrode are not particularly limited as long as they have excellent conductivity. For example, metals such as copper, aluminum, stainless steel, gold, silver, and platinum and their plated or deposited materials, conductive carbon materials such as carbon black, graphite, and carbon nanotubes and their composite materials mixed with resins, or the like are usable. The material constituting the first electrode and the material constituting the second electrode may be the same or different from each other.

Among these materials, aluminum, stainless steel, or silver is preferable as the material constituting the first electrode from the viewpoint of avoiding the corrosion of the electrode and achieving uniform discharge. For the same reason, aluminum, stainless steel, or silver is preferable as the material constituting the second electrode.

Further, the first electrode and the second electrode may take the form of a flat plate, wire, needle, or the like without any restriction. The first electrode preferably takes the form of a flat plate. Further, the second electrode preferably takes the form of a flat plate. In a case where at least one of the first electrode and the second electrode takes the form of a flat plate, the flat plate preferably has an aspect ratio (the length of the long side/the length of the short side) of at least 2.

A material constituting the dielectric is not particularly limited as long as it has high electric insulating performance. Resins such as polyimide, polyester, fluorocarbon resin, silicone resin, acrylic resin, and phenol resin, glass, ceramics, and their composite materials mixed with resins, or the like are, for example, usable. Among these materials, ceramics, glass, or silicone resin is suitably used as the dielectric from the viewpoint of strength and insulating performance. Particularly, a silicone resin is capable of enhancing the freedom of degree in the shape of the plasma actuator due to its flexibility.

### Plasma Actuator

The plasma actuator is not particularly limited as long as the first electrode and the second electrode are provided via the dielectric and an induced flow representing a unidirectional jet flow containing ozone is capable of being generated by the application of a voltage between both the electrodes.

In the plasma actuator, plasma is more easily generated as the shortest distance between the first electrode and the second electrode is smaller. Therefore, the thinner the film thickness of the dielectric within the range where electric insulation breaks down does not occur, the more preferable it is. The film thickness may be in the range from 10 μm to 1,000 μm and preferably in the range from 10 μm to 200 μm. Further, the shortest distance between the first electrode and the second electrode is preferably not more than 200 μm.

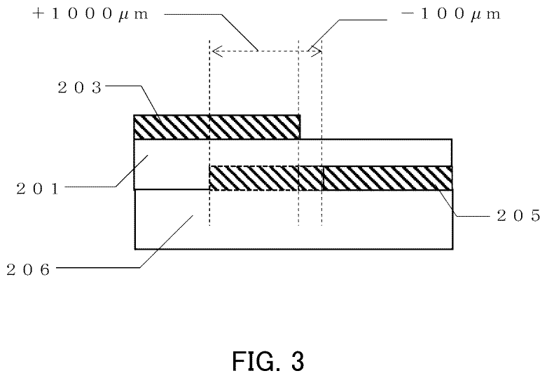

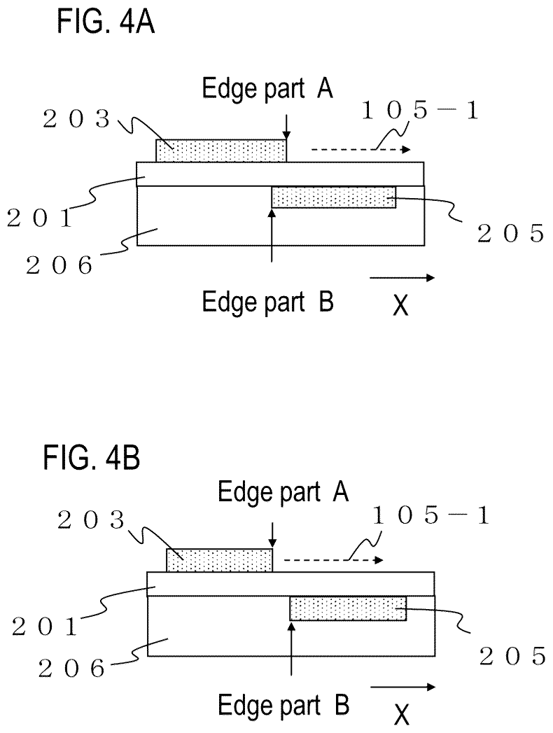

FIGS. 3, 4A, and 4B are explanatory views of the overlap between the first electrode 203 and the second electrode 205 of the plasma actuator serving as an ozone generation device. FIGS. 3, 4A, and 4B are cross-sectional views of the plasma actuator.

When the plasma actuator is viewed through from the side of the first electrode (first surface), the first electrode 203 and the second electrode 205 arranged diagonally opposite to each other are such that the edge part of the first electrode may be present in the formed area of the second electrode via the dielectric. For example, the first electrode and the second electrode may be provided to overlap each other in the Y-axis direction via the dielectric. In this case, it is preferable to prevent insulation from breaking down in the area, where the first electrode and the second electrode overlap each other via the dielectric, during the application of a voltage.

FIG. 4A shows an aspect in which the first electrode and the second electrode overlap each other (in the Y-axis direction) via the dielectric. In the cross section of the plasma actuator in the thickness direction, it is assumed that the edge part of the first electrode on the side of the first direction is an edge part A, and that the edge part of the second electrode on the side of a second direction (opposite to the X-direction) opposite to the first direction is an edge part B. At this time, the edge part B is preferably positioned on the side of the second direction (opposite to the X-direction) relative to the edge part A.

Further, assuming that the edge part of the first electrode on the side of the first direction is an edge part A and the edge part of the second electrode on the side of the second direction (opposite to the X-direction) opposite to the first direction is an edge part B when the plasma actuator is viewed through from the side of the first electrode, the edge part B is preferably positioned on the side of the second direction (opposite to the X-direction) relative to the edge part A.

The first electrode and the second electrode overlap each other via the dielectric as described above, thus enabling stable occurrence of plasma and an induced flow.

Further, since the first electrode and the second electrode are arranged diagonally opposite to each other via the dielectric 201, the edge part B is positioned on the side of the first direction (X-direction) relative to the edge part of the first electrode on the side opposite to the edge part A. Thus, the occurrence of an induced flow from the edge part of the first electrode on the side opposite to the edge part A is suppressed.

FIG. 4B shows an aspect in which the first electrode and the second electrode do not overlap each other (in the Y-axis direction) via the dielectric. Assuming that the edge part of the first electrode on the side of the first direction is an edge part A and the edge part of the second electrode on the side of the second direction (opposite to the X-direction) opposite to the first direction is an edge part B in the cross section of the plasma actuator in the thickness direction, the edge part B is preferably positioned on the side of the first direction (the X-direction) relative to the edge part A.

Further, assuming that the edge part of the first electrode on the side of the first direction is an edge part A and the edge part of the second electrode on the side of the second direction (opposite to the X-direction) opposite to the first direction is an edge part B when the plasma actuator is viewed through from the side of the first electrode, the edge part B is preferably positioned on the side of the first direction (opposite to the X-direction) relative to the edge part A.

In a case where the first electrode and the second electrode do not overlap each other via the dielectric as described above, it is preferable to relatively increase the voltage applied between both the electrodes in order to compensate for the weakening of an electric field caused when the shortest distance between the electrodes relatively becomes large.

Further, assuming that the edge part of the first electrode on the side of the first direction is an edge part A and the edge part of the second electrode on the side of the second direction (opposite to the X-direction) opposite to the first direction is an edge part B when the plasma actuator is viewed through from the side of the first electrode, the edge part A and the edge part B preferably align with each other in the thickness direction (the Y-axis direction) of the dielectric as another aspect. Further, the edge part A and the edge part B preferably align with each other in the thickness direction (the Y-axis direction) of the dielectric in the cross section of the plasma actuator in the thickness direction as another aspect. This aspect refers to, for example, an aspect in which the edge part A and the edge part B are opposed to each other at the shortest distance via the dielectric, and the first electrode and the second electrode either do not overlap each other via the dielectric or do not separate from each other. Thus, the energy applied between both the electrodes is efficiently used to generate an induced flow.

Assuming that an overlapping length is considered positive, the overlap between the edge part A of the first electrode and the edge part B of the second electrode is preferably in the range from −100 μm to +1,000 μm, more preferably in the range from −0 μm to +200 μm, and still more preferably at 0 μm in the X-axis direction when viewed from the top of the cross-sectional view (FIG. 3). That is, assuming that a case where the edge part B is positioned on the side opposite to the blowing-out direction of an induced flow relative to the edge part A is considered positive, the interval between the edge part A and the edge part B in the direction (the X-axis direction) along the surface of the dielectric is preferably in the range from −100 μm to +1,000 μm, more preferably in the range from 0 μm to +200 μm, and still more preferably at 0 μm. However, it is difficult to achieve a constant overlap of 0 μm over the Z-axis direction from the viewpoint of machining accuracy in manufacturing the plasma actuator. Accordingly, it is common practice to provide a positive overlap corresponding to machining accuracy.

The thicknesses of both the first electrode and the second electrode are not particularly limited but may be in the range from 10 μm to 1,000 μm. When the thicknesses of the electrodes are at least 10 μm, the resistances of the electrodes become low, making it easier for plasma to be generated. When the thicknesses of the electrodes are not more than 1,000 μm, electric field concentration occurs more easily, making it easier for plasma to be generated.

The widths of both the first electrode and the second electrode are not particularly limited but may be at least 1,000 μm.

The shapes of the electrodes are not particularly limited but are preferably, for example, a rectangle such as an oblong and a square. The electrodes having a rectangle enable the generation of a uniform induced flow.

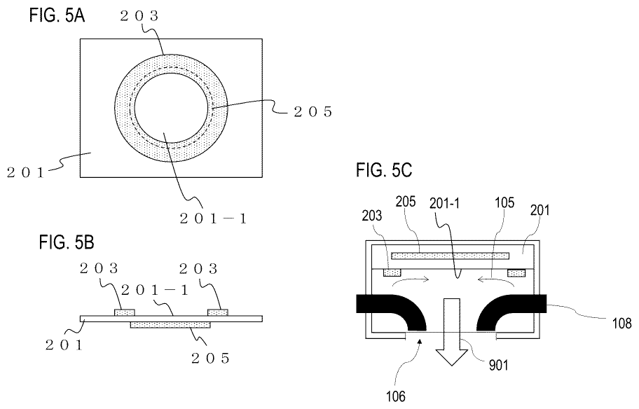

Further, as shown in FIGS. 5A (a perspective view from the side of the first surface of the dielectric) and 5B (a cross-sectional view of the plasma actuator in the thickness direction), the first electrode may have a doughnut shape, and the second electrode may have a circular or doughnut shape. Even with such electrode shapes, the first electrode 203 and the second electrode 205 are arranged diagonally opposite to each other via the dielectric 201 in the thickness direction of the plasma actuator when viewed in cross section in the thickness direction. Further, the first electrode 203 is provided to coat a part of the first surface of the dielectric 201, and the first surface has the exposed part 201-1 not coated with the first electrode 203. Moreover, when the plasma actuator is viewed through from the side of the first surface (FIG. 5A), at least a part of the exposed part 201-1 of the dielectric and the second electrode 205 overlap each other (at a hole in the doughnut-shaped first electrode).

Even in such a doughnut-shaped electrode DBD-PA, the application of a voltage between the first electrode and the second electrode results in the generation of a dielectric barrier discharge oriented from the first electrode toward the second electrode. As a result, an induced flow blows out in one direction along the surface of the dielectric from the first electrode. The blown-out induced flow collides near the center of the electrode and transforms into a three-dimensional Wall normal jet ejected upward in FIG. 5B.

An example of an active oxygen supply device using such a doughnut-shaped electrode DBD-PA is shown in FIG. 5C. In the active oxygen supply device shown in FIG. 5C, the induced flow 105 containing ozone is ejected from the edge part on the side of the inner periphery of the doughnut-shaped first electrode toward the first direction representing one direction along the surface of the dielectric 201, that is, toward a central direction. Then, the induced flow 105 collides at the central part of the first electrode, thus generating an axis-symmetric jet flow 901 containing the ozone toward the direction perpendicular to the surface (exposed part) 201-1 of the dielectric 201 (toward a vertically lower side in FIG. 5C). Then, the axis-symmetric jet flow 901 is humidified by the humidifying device (not shown) to decompose the ozone contained in the axis-symmetric jet flow into active oxygen, and the axis-symmetric jet flow containing the active oxygen flows to the outside of the housing 107 from the opening part 106. Then, an object to be treated, which is present near the opening part 106, is treated by the active oxygen contained in the axis-symmetric jet flow 901 flowing out from the opening part 106.

Further, the plasma actuator may also be a so-called three-electrode plasma actuator in which a third electrode is additionally provided on a downstream side in the blowing-out direction of an induced flow from the first electrode and on the first surface of the dielectric 201. In this case, for example, it is possible to apply an AC voltage using the first electrode as an AC electrode, and apply a DC voltage using the third electrode as a DC electrode. It is also possible to generate a sliding discharge by applying a negative DC voltage to the DC electrode.

Further, in a case where the edge part of the second electrode is exposed, plasma may be generated also from the edge part of the second electrode, potentially resulting in the generation of an induced flow in the direction opposite to the induced flow 105 derived from the first electrode. In the active oxygen supply device according to this aspect, it is preferable to minimize ozone concentration in the inner space of the active oxygen supply device, excluding the surface region of an object to be treated, as much as possible. Further, it is preferable to prevent the generation of an airflow that disrupts the induced flow 105 in a container. Therefore, it is preferable to prevent the generation of an induced flow derived from the second electrode.

In view of this, the second electrode 205 is preferably an embedded electrode so that plasma is not generated from the second electrode 205. For example, as shown in FIGS. 2A, 2B and 3, the second electrode may be coated with a dielectric such as the dielectric substrate 206, or embedded in the dielectric 201. The second electrode may be embedded to such a degree that the generation of plasma from the edge part of the second electrode is prevented. For example, a part of the surface of the second electrode may be exposed, and the exposed surface of the second electrode and the dielectric substrate 206 or the dielectric 201 may form the same plane. The edge part of the second electrode is preferably coated with the dielectric substrate 206 or the dielectric 201.

Accordingly, the plasma actuator is preferably, for example, a single dielectric barrier discharge (SDBD) plasma actuator.

In the plasma actuator, an induced flow is not preferably generated from edge parts other than the edge part A of the first electrode defined as described above. Therefore, the edge parts other than the edge part A may be coated with the dielectric. In this manner, a unidirectional jet flow is generated even when the first electrode and the second electrode overlap each other in the Y-axis direction. Further, the shapes of the electrodes may be controlled so that an induced flow is not generated from the edge parts other than the edge part A according to the relationship between the first electrode and the second electrode. For example, in a case where the electrodes have the form of a rectangle, the first electrode and the second electrode may have the same length or the first electrode may be longer than the second electrode in the Z-axis direction (the direction perpendicular to the blowing-out direction of an induced flow from the edge part A). According to such an aspect, the direction of an induced flow is actively easily controlled.

The induced flow 105 containing concentrated ozone flows in the flowing direction of a jet-like flow generated by surface plasma along the exposed part 201-1 of the first surface of the dielectric 201 from the edge part 204 of the first electrode 203, that is, in the direction along the exposed part 201-1 of the first surface of the dielectric from the edge part 204 of the first electrode 203. This induced flow is a flow of gas containing concentrated ozone with a speed of approximately several meters per second to several tens of meters per second.

The voltage applied between the first electrode 203 and the second electrode 205 of the plasma actuator is not particularly limited as long as it enables the plasma actuator to generate plasma. Further, a DC voltage or an AC voltage may be applied, but the AC voltage is preferably applied. Further, a pulse voltage may be preferably applied as the voltage.

Moreover, the amplitude and frequency of the voltage may be appropriately set in order to adjust the flow velocity of an induced flow and ozone concentration in the induced flow. In this case, the amplitude and frequency of the voltage may be appropriately selected from the viewpoint of generating ozone concentration, which is necessary for generating an effective active oxygen concentration or effective active oxygen amount according to the purpose of treatment, in the induced flow, supplying generated active oxygen to the surface region of an object to be treated while maintaining an effective active oxygen concentration or effective active oxygen amount according to the purpose of treatment, or the like.

For example, the amplitude of the voltage may be in the range from 1 kV to 100 kV. Furthermore, the frequency of the voltage may be preferably at least 1 kHz and more preferably in the range from 10 kHz to 100 KHz.

In a case where the voltage is an AC voltage, the waveform of the AC voltage is not particularly limited, and a sine wave, a rectangular wave, a triangular wave, or the like may be employed. However, the rectangular wave is preferable from the viewpoint of a fast rising voltage.

The duty ratio of the voltage is also appropriately selectable, but the voltage preferably rises fast. Preferably, the voltage is applied so that the rate of voltage rise, which reaches from the bottom to the top of the amplitude of a wavelength, becomes at least 10,000,000 V per second.

Note that a value obtained by dividing the amplitude of the voltage applied between the first electrode 203 and the second electrode 205 by the film thickness of the dielectric 201 (voltage/film thickness) is preferably at least 10 kV/mm.

### Humidifying Device

The humidifying device is not particularly limited as long as it is capable of humidifying the inside of the housing to cause water to be contained in an induced flow and decomposing ozone in the induced flow with the water to generate active oxygen in the induced flow. Here, the humidifying process involves giving moisture to a target, and the form of the moisture is not particularly limited but may be at least one selected from a group including gas, a liquid, and a solid. Further, known water is arbitrarily usable as water for giving moisture, and this water may contain substances other than water.

As an aspect of the humidifying device, the humidifying device is composed of a humidified air generation unit, an air blowing unit, a flow rate adjustment valve, a flow rate measurement unit, a temperature/humidity measurement unit, an emission unit that emits humidified air, and pipes that connect the respective configuration unit together. Such an aspect enables the humidification of the inside of the housing, thereby causing water to be contained in an induced flow containing ozone.

The humidifying device is not particularly limited to the above aspect but is, for example, a vaporizing type humidifying device or a mist type humidifying device.

In order to suppress humidity near the plasma actuator, the humidifying device is preferably one that has directivity with respect to a moisture supply direction (that will also be simply called directivity below). With the directivity of the humidifying device, it is possible to efficiently humidify the vicinity of an induced flow or the vicinity of the surface of an object to be treated without increasing humidity near the plasma actuator.

In order to make the humidifying device have the directivity, a known method is suitably available. For example, a method in which an airflow is generated by a fan and moisture is supplied to the direction of the airflow, a method in which an appropriate pressure is given to moisture by an air pump or the like to inject the moisture in a desired direction, or the like, is available. In order to prevent an induced flow from being disrupted, the humidifying device is preferably directed in the same direction (first direction) as the induced flow.

The degree of humidification by the humidifying device is not particularly limited as long as it humidifies the inside of the housing to cause water to be contained in an induced flow and decomposes ozone in the induced flow by the water to generate active oxygen in the induced flow. Relative humidity at the opening part is preferably at least 60% RH, more preferably at least 80% RH, and still more preferably at least 90% RH. Further, the relative humidity is generally not more than 100% RH, and may be not more than 95% RH.

### Arrangement of Plasma Actuator, Humidifying Device, and Object to Be Treated

In the active oxygen supply device 101, the position of the plasma actuator 103 that generates an induced flow containing ozone is not particularly limited as long as the plasma actuator 103 is arranged so that the induced flow 105 generated by moisture from the humidifying device flows to the outside of the housing from the opening part and is supplied to the surface of an object to be treated while maintaining an effective active oxygen concentration or effective active oxygen amount according to the purpose of treatment.

For example, the plasma actuator and the humidifying device may be arranged so that the induced flow 105 containing active oxygen generated by moisture is supplied to the surface of an object to be treated at the shortest distance.

Further, for example, an object to be treated may be arranged so that the treatment surface 104-1 of the object is positioned on the extension line in the direction along the exposed part 201-1 of the first surface of the dielectric from the edge part 204 of the first electrode 203 of the plasma actuator on the side of the first direction. For example, the extension line preferably touches the treatment surface 104-1.

Further, the extension line in the direction (the X-direction) along the first surface of the dielectric from the edge part of the first electrode 203 of the plasma actuator on the side of the first direction is preferably directed to the opening part. Thus, an induced flow easily flows to the outside of the housing from the opening part.

Moreover, in a case where the opening part of the active oxygen supply device is directed vertically downward, the narrow angle formed between the extension line in the direction along the exposed part 201-1 of the first surface of the dielectric from the edge part of the first electrode of the plasma actuator and a horizontal plane (a plane perpendicular to a vertical direction) is designated as θ. The narrow angle θ is not particularly limited as long as it enables an induced flow to be actively supplied to the surface region of an object to be treated or enables the induced flow to be treated by active oxygen while maintaining an effective active oxygen concentration or effective active oxygen amount according to the purpose of treatment, but is preferably from 0° to 90°.

By the arrangement of the plasma actuator and the humidifying device as described above, it is possible to locally supply an induced flow having a certain flow velocity and containing active oxygen to a region near the surface of an object to be treated, or possible to treat the object with the active oxygen. Further, an induced flow flowing out from the opening flows along the surface of an object to be treated, thereby exposing portions other than a portion opposed to the opening part in the treatment surface of the object to the induced flow containing active oxygen. In this manner, it is possible to treat a wider range of the surface to be treated (treatment surface) 104-1 with active oxygen.

Further, the plasma actuator may be arranged so that the treatment surface 104-1 of an object to be treated is positioned on the extension line of the first direction (the blowing-out direction of an induced flow). In a case where the opening part of the active oxygen supply device is directed vertically downward, the narrow angle formed between the first direction (the blowing-out direction of an induced flow) and a horizontal place (a plane perpendicular to a vertical direction) is designated as θ′. The angle θ′ is preferably in the range from 0° to 90°.

The arrangement of the humidifying device is not particularly limited as long as the humidifying device is arranged to be capable of humidifying the inside of the housing to humidify an induced flow and generate active oxygen in the induced flow and performing treatment on the surface of an object to be treated while maintaining an effective active oxygen concentration or effective active oxygen amount according to the purpose of treatment. For example, the entire humidifying device may be arranged inside of the housing. Alternatively, only a portion constituting the humidifying device may be connected to the housing, and humidification may be performed at the connected point. As an aspect of the connected point, for example, an emission part that emits humidified air in the portion constituting the humidifying device is connected to the housing.

Hereinafter, a description relating to the arrangement of the humidifying device refers also to a description relating to the connected point.

As described above, an induced flow containing ozone is actively supplied to a region near the surface of an object to be treated. Further, it is possible to humidify the induced flow to generate active oxygen in the induced flow. Therefore, as the induced flow is humidified, the ozone is excited, thus enabling the induced flow where the active oxygen has been generated to be actively supplied to the surface of the object and a significant increase in an active oxygen concentration or active oxygen amount on the surface of the object.

The relative position of the humidifying device and the plasma actuator is not particularly limited as long as the humidifying device and the plasma actuator are arranged to be capable of generating active oxygen in an induced flow and performing treatment on the surface of an object to be treated while maintaining an effective active oxygen concentration and effective active oxygen amount according to the purpose of the treatment.

Further, regarding the distance between the humidifying device and the plasma actuator, the position of the humidifying device with respect to the plasma actuator may be set so that an induced flow is humidified to generate active oxygen in the induced flow, and that the induced flow containing an effective amount of the active oxygen according to the purpose of treatment flows to the outside of the housing from the opening part to be supplied to an object to be treated. As an example of the arrangement position of the humidifying device with respect to the plasma actuator, the distance between the opposing surface of the dielectric of the plasma actuator and the humidifying device is preferably not more than 10 mm and more preferably not more than 4 mm. However, it is not necessary to arrange the plasma actuator at a place within approximately 10 mm from the humidifying device. The distance between the humidifying device and the plasma actuator is not particularly limited as long as active oxygen in an induced flow has an effective concentration according to the purpose of treatment in consideration of moisture or the like supplied from the humidifying device.

Further, at least one of the humidifying device and the plasma actuator is preferably provided with a moving unit as an aspect so that at least one of the humidifying device and the plasma actuator is made freely movable to enable uniform humidification.

In the relative position of the active oxygen supply device and an object to be treated, at least one of the active oxygen supply device and the object may be arranged so that active oxygen is generated in an induced flow and the surface of the object is exposed to the induced flow maintaining an effective active oxygen concentration and effective active oxygen amount according to the purpose of treatment.

Further, the humidifying device may be arranged at a position where the surface of an object to be treated is capable of being humidified, or may be arranged at a position where the surface of an object to be treated is not capable of being humidified. Even in a case where the surface of an object to be treated is not capable of being humidified, the treatment device using active oxygen according to this aspect enables treatment by exposing a treatment surface to active oxygen in an induced flow. Moreover, sterilization treatment using the active oxygen supply device according to the present disclosure enables the sterilization of funguses present at positions where active oxygen reaches. Accordingly, for example, it is possible to sterilize even funguses present between fibers.

On the other hand, in a case where the humidifying device is arranged to be capable of humidifying the surface of an object to be treated, which is positioned outside of the housing, via the opening part, the humidifying device is capable of decomposing undecomposed ozone present in an induced flow in situ on a treatment surface and generating active oxygen on the treatment surface. As a result, it is possible to further increase a treatment degree or treatment efficiency.

Moreover, the distance between the humidifying device and the surface of an object to be treated may be adjusted according to the purpose of treatment and is not particularly limited. However, taking the lifespan of active oxygen contained in an induced flow into consideration, it is preferable to set the distance at not more than 10 mm and more preferable to set the distance at not more than 4 mm. However, it is not necessary to arrange an object to be treated so that its surface is positioned at a place within approximately 10 mm from the humidifying device. The distance between the humidifying device and an object to be treated is not particularly limited as long as active oxygen in an induced flow has an effective concentration according to the purpose of treatment in consideration of a humidification method or the like.

In addition, the relative positions of the humidifying device, the plasma actuator, and the opening part may be arranged so that active oxygen is generated in an induced flow, and that the surface of an object to be treated is exposed to the induced flow maintaining an effective active oxygen concentration or effective active oxygen amount according to the purpose of treatment.

Considering that the generation amount of ozone decreases in the presence of high moisture near the surface plasma 202 and taking the lifespan of active oxygen contained in an induced flow into consideration, it is preferable to install the humidifying device on the downstream side of the induced flow generated by the plasma actuator. That is, the plasma actuator, the humidifying device, and the opening part are preferably arranged in this order toward the longitudinal direction inside of the housing.

Further, in the plasma actuator, an ozone generation amount per unit time in a state in which an induced flow is not humidified is, for example, preferably at least 8 μg/min. The ozone generation amount is more preferably at least 15 μg/min. An upper limit of the ozone generation amount is not particularly limited but is, for example, not more than 1,000 μg/min. That is, the ozone generation amount is preferably in the range from at least 8 μg/min to not more than 1,000 μg/min.

The flow velocity of an induced flow may be, for example, a velocity at which generated active oxygen is capable of being actively supplied to the surface region of an object to be treated while maintaining an effective active oxygen concentration or effective active oxygen amount according to the purpose of treatment. The flow velocity is, for example, in the range from approximately 0.01 m/s to 100 m/s as described above.

The concentration of ozone in an induced flow generated from the plasma actuator as described above or the flow velocity of the induced flow may be controlled by the thicknesses or materials of the electrodes or the dielectric, the type, amplitude, frequency of an applied voltage, or the like.

From the viewpoint of suppressing humidity near the plasma generation device 103 and efficiently generating ozone, a blocking member that blocks moisture to the plasma generation device may be provided between the plasma generation device 103 and the humidifying device. The blocking member may have a member to prevent the permeation of moisture on its humidifying device side. Known materials are suitably usable as the member to prevent the permeation of moisture. Examples of such materials include a metal tape containing metals such as aluminum, copper, and stainless steel, a metal film, a metal plate, and resin such as polyethylene, polypropylene, polyvinyl alcohol, and ethylene vinyl alcohol copolymer. The blocking member is preferably provided at a position at which the blocking member does not hinder an induced flow. Specifically, the blocking member is preferably not positioned on the extension line in the direction along the surface of the dielectric 201 from the edge part of the first electrode 203 of the plasma actuator. Further, in order to decrease humidity near the plasma generation device 103, the plasma generation device may have a dehumidifying mechanism or a dehumidifying agent.

### Housing and Opening Part

The active oxygen supply device according to the present disclosure comprises: the housing 107 having the at least one opening part 106; the humidifying device arranged inside of the housing; and the plasma actuator 103.

The opening part is not particularly limited as long as the induced flow 105 generated from the plasma actuator 103 flows to the outside of the housing 107. The size of the opening part, the position of the opening part, and the relative position of the opening part and an object to be treated may be appropriately selected sot that, for example, generated active oxygen is capable of being actively supplied to the surface region of the object while maintaining an effective active oxygen concentration or effective active oxygen amount according to the purpose of treatment.

Moreover, the distance between the plasma actuator and the opening part is preferably short in order to more effectively use active oxygen in an induced flow for intended treatment. Therefore, the plasma actuator is preferably arranged at a position closer to the opening part. On the other hand, the plasma actuator is preferably arranged at a position set back from the opening part for its protection. As an example, the plasma actuator is preferably arranged on the inner wall of the housing so that the end of the plasma actuator on the side close to the opening part is positioned 0.5 mm to 1.5 mm away from the end of the opening part of the inner wall of the housing. The housing may have an air intake part 109 as an air inflow port. Due to the air intake part 109, when an induced flow is generated by the plasma actuator 103 and gas inside of the housing moves toward the opening part, air flows in from the outside of the air intake part 109, thus enabling the generation of an airflow resulting from the induced flow, which is oriented from the air intake part 109 toward the opening part 106, inside of the housing.

The active oxygen supply device according to the present disclosure is applicable not only to the sterilization of an object to be treated but also to various applications implemented by supplying active oxygen to an object to be treated. For example, the active oxygen supply device according to the present disclosure is applicable also to purposes such as deodorization, bleaching, and hydrophilic surface treatment of an object to be treated.

Further, the treatment device using active oxygen according to the present disclosure is used not only for treatment to sterilize an object to be treated but also for other purposes such as treatment to deodorize an object to be treated, treatment to bleach an object to be treated, and surface treatment to make an object to be treated hydrophilic.

Further, the present disclosure provides a treatment method for treating the surface of an object to be treated using active oxygen, the treatment method comprising:
a step of preparing the above treatment device using active oxygen;
a step of arranging the prepared treatment device using the active oxygen and the object at relative positions so that the surface of the object to be treated is exposed when an induced flow containing the active oxygen flows out from the opening part; and
a step of causing the induced flow containing the active oxygen to flow out from the opening part to treat the surface of the object using the active oxygen.

Note that the “effective active oxygen concentration or effective active oxygen amount” in the present disclosure refers to an active oxygen concentration or active oxygen amount for achieving purposes such as sterilization, deodorization, and hydrophilization for an object to be treated, and may be appropriately adjusted according to purposes using the thicknesses and materials of the electrodes and the dielectric constituting the plasma actuator, the type, amplitude, and frequency of an applied voltage, the moisture amount and humidification time of humidification, or the like.

The active oxygen supply device according to the present disclosure is capable of performing treatment while moving at least one of the active oxygen treatment device and an object to be treated, for example, when the area of the treatment surface of the object is larger than the opening. At this time, the relative movement speed or movement direction of the active oxygen supply device and the object may be appropriately set as long as the treatment surface is capable of being treated to a desired degree and are not particularly limited. Similarly, the number of the treatment times of the object may be appropriately set as long as the treatment surface is capable of being treated to a desired degree.

### EXAMPLES

The present disclosure will be described in further detail using Examples and Comparative Examples below, but the aspect of the present disclosure is not limited to the Examples and Comparative Examples.

### Example 1

### 1. Humidifying Device

A humidifying device according to this Example was composed of a humidified air generation unit, an air blowing unit, a flow rate adjustment valve, a flow rate measurement unit, a temperature/humidity measurement unit, an emission unit that emits humidified air, and pipes that connect the respective configuration unit together. In the humidified air generation unit, an ultrasonic sprayer (manufactured by REN HE Company) was operated to generate humidified air in a petri dish filled with water. In the air blowing unit, a suction pump (LIQUIPORT NF-100KT. 18S, manufactured by Yamato Scientific Co., Ltd.) was used. In the flow rate measurement unit, an amplifier-separated gas flow-rate sensor FD-V40 series (manufactured by KEYENCE CORPORATION) was used as a flow rate meter. In the emission unit, a silicone tube having an inner diameter of 4 mm (an outer diameter of 6 mm) was used as a pipe. The air blowing unit, the humidified air generation unit, the flow rate adjustment valve, the flow rate measurement unit, the temperature/humidity measurement unit, and the emission unit were connected in this order, and used as units to control the gas flow rate of a pump to adjust the humidity of an active oxygen supply device, which will be disclosed below, within the range where a gas flow velocity at the emission unit was substantially smaller than the flow velocity of an induced flow generated by a plasma actuator and ranged from 0.0 m/sec to 0.5 m/sec, ensuring that the induced flow was not disrupted.

### 2. Manufacturing of Active Oxygen Supply Device

An aluminum foil having a longitudinal length of 2.5 mm, a horizontal length of 15 mm, and a thickness of 100 μm was pasted onto a first surface of a glass plate (having a longitudinal length of 5 mm, a horizontal length (a depth direction in FIGS. 1A and 1B) of 18 mm, and a thickness of 150 μm) serving as a dielectric, using an adhesive tape to form a first electrode. Further, an aluminum foil having a longitudinal length of 3 mm, a horizontal length of 15 mm, and a thickness of 100 μm was pasted onto a second surface of the glass plate using an adhesive tape so as to be diagonally opposite to the aluminum foil pasted onto the first surface to form a second electrode. Moreover, the second surface including the second electrode was coated with a polyimide tape. In this manner, a plasma actuator was manufactured with the first electrode and the second electrode overlapping each other over a width of 0.5 mm via the dielectric (glass plate).

Next, as a housing 107 of an active oxygen supply device 101, a rectangular housing made of an ABS resin and having a penetration hole was prepared. The cross-sectional shape of the housing is shown in FIG. 1A. The housing had a height of 30 mm (in a vertical direction in FIG. 1A), a width of 11 mm (in a horizontal direction in FIG. 1A), a length of 34 mm (in a depth direction in FIG. 1A), and a thickness of 2 mm between the inner wall and outer wall of the penetration hole. The case of the housing had an opening part 106 having a width of 7 mm and a length of 30 mm as a blowing-out part for an induced flow on one surface and an air intake part 109 having a width of 7 mm and a length of 30 mm on the side opposite to the opening part 106. Next, an edge part 204 of the first electrode 203 of the one previously-manufactured plasma actuator was fixed to overlap a spot 15 mm representing the midpoint of the 30 mm height of the inner wall of the housing 107. Specifically, as shown in FIG. 1A, the plasma actuator 103 was fixed so that the direction (X-direction) along an exposed part 201-1 of the first surface of a dielectric 201 was oriented toward the side of the opening part, and that the angle (equivalent to θ) formed at the intersecting point between an extension line in the direction along the exposed part 201-1 of the first surface of the dielectric 201 and the treatment surface of an object 104 to be treated was set at 90°.

Moreover, an emission unit 108 of the above humidifying device (not shown) was arranged at the position shown in FIG. 1A inside of the housing to serve as the connected spot between the housing and the humidifying device. Specifically, the distance (denoted by symbol 108-1 in FIG. 1A) between the center of the emission unit 108 having a diameter Φ of 6 mm and the edge part 204 of the first electrode 203 of the plasma actuator was set at 10 mm. At the time, the distance between the center of the emission unit 108 and the opening part 106 was 5 mm.

Subsequently, the active oxygen supply device 101 was placed into a sealed container (not shown) having a volume of 1 L to calculate the amount of ozone generated from the plasma actuator 103. The sealed container was equipped with an opening sealable with a rubber plug, thus enabling the suction of internal gas from the opening using an injector. Then, the humidifying device was not operated, and an AC voltage with a sine waveform having an amplitude of 2.4 kVpp and a frequency of 80 kHz was applied to the plasma actuator 103. After 1 minute had elapsed, 100 mL of the gas inside of the sealed container was collected. The collected gas was sucked by an ozone detection tube (product name: 182SB, manufactured by KOMYO RIKAGAKU KOGYO K.K.) to measure the concentration (PPM) of measured ozone contained in an induced flow from the plasma actuator 103. Using the value of the measured ozone concentration, an ozone generation amount per unit time was calculated by the following equation.

$$Amount of ozone generated per unit time ( \frac{mg}{min} ) = Measured ozone concentration ( PPM ) * \frac{Molecular weight of ozone 48}{22.4} * \frac{\frac{273}{273 + Room temperature ( ° C . )}}{10000} * \frac{Gas inside of sealed container ( L )}{Collected gas ( L )} = Measured ozone concentration ( PPM ) * 48 / 22.4 * 273 / ( 273 + 25 ) / 10000 * 0.1 / 1$$

As a result, the ozone generation amount per unit time was 18 μg/min. At this time, the humidifying device was not powered on to prevent an affect by ozone decomposition due to humidified air.

Finally, the ozone generation amount during the operation of both the plasma actuator 103 and the humidifying device was measured. The operating condition of the plasma actuator 103 was such that 18 μg/min of ozone was generated when only the plasma actuator 103 was operated. Further, the humidifying device was adjusted to have a relative humidity of 95% RH (a temperature of 25° C.) at the exit of the emission unit when the gas flow rate of the pump was controlled within the range where the gas flow velocity at the emission unit ranged from 0.0 m/sec to 0.5 m/sec. At this time, humidity was measured using a digital thermohygrometer (product name: CHF-TP1, manufactured by SANWA SUPPLY INC.). Then, both the plasma actuator 103 and the humidifying device were operated. As a result of the measurement of humidity at each of the opening part 106 and the air intake part 109, it was found that the humidity was 90% RH at the opening part 106 and 60% RH at the air intake part 109. At this time, the ozone generation amount was 7 μg/min. It appears that 11 μg/min, which corresponds to the reduction amount from 18 μg/min, represents the amount of ozone transformed into active oxygen.

### 2-1. Detection Test for Active Oxygen (Absorbance of Methylene Blue)

The presence or absence of active oxygen in an airflow flowing out from the opening part 106 was confirmed by making use of the decolorization of methylene blue (see “Magnetic Field Effect on the Photocatalytic Reaction with TiO2 Semiconductor Film,” Masanobu Wakasa et al., Journal of The Society of Photographic Science and Technology of Japan, 69, 4, 271-275 (2006)). Methylene blue is crystalline powder with a blue luster and is soluble in water or ethanol. Therefore, methylene blue is used in a solution state as a dye or an indicator. Then, methylene blue reacts with active oxygen to undergo decomposition and loses its blue color. Therefore, the presence or absence of active oxygen in an induced flow is confirmable by the decolorization (loss of blue color) of methylene blue.

Specifically, the following operation was conducted. Methylene blue (produced by KANTO CHEMICAL CO., INC., highest quality) was mixed with distilled water to prepare a 0.01% methylene blue solution. 15 mL of the methylene blue solution was placed into a petri dish (AB4000, manufactured by EIKEN CHEMICAL CO., LTD., a column shape having a diameter of 88 mm). As a result, a petri dish A containing the methylene blue solution was prepared. The active oxygen supply device 101 and the petri dish A were arranged so that the surface center of the petri dish A and the center of the opening part 106 of the active oxygen supply device 101 were opposed to each other at a distance of 1 mm.

Subsequently, the humidifying device was operated under the same conditions (with a relative humidity of 95% RH (and a temperature of 25° C.) at the exit of the emission unit), while applying an AC voltage with a sine waveform having an amplitude of 2.4 kVpp and a frequency of 80 kHz between both electrodes of the active oxygen supply device 101, thereby supplying an induced flow flowing out from the opening to the surface of the liquid for 120 minutes and treating the methylene blue solution.

The methylene blue solution after the supply of the induced flow was transferred from the petri dish to a cell, and a change in the light absorption amount of methylene blue was measured using a spectrophotometer (product name: V-570, manufactured by JASCO Corporation). Since methylene blue exhibits strong absorption at a wavelength of 664 nm, the degree of the decolorization of methylene blue is calculatable from a change in absorbance at the wavelength. In this test, when only distilled water was first placed into the reference cell and a 0.01% methylene blue solution before the supply of the induced flow was placed into the sample cell and measured, the absorbance was 2.32 Abs. On the other hand, the absorbance of the methylene blue solution after the supply of the induced flow was 0.19 Abs. Accordingly, the reduction rate of the absorbance was ((2.32−0.19)/2.32)×100=92%.

### 2-2. Treatment (Sterilization) Test

Using the active oxygen supply device 101, a sterilization test for E.coli was conducted in the following procedure. Note that all the equipment used in this sterilization test were those having undergone high-pressure steam sterilization using an autoclave. Further, this sterilization test was conducted inside of a clean bench.

First, E. coli (product name “KWIK-STIK (E. coli (Escherichia coli) ATCC8739)”, produced by Microbiologics, Inc.) was placed into an Erlenmeyer flask containing an LB medium (prepared by dissolving a mixture of 2 g of tryptone, 1 g of yeast extract, and 1 g of sodium chloride in distilled water to make a total volume of 200 mL), and the Erlenmeyer flask was shaken and incubated for 48 hours at 37° C. and at 80 rpm. An E. coli solution after the incubation was 9.2×10⁹ (CFU/mL).

0.010 mL of the E. coli solution after the incubation was dispensed onto a qualitative filter paper (product number: No. 5C, manufactured by Advantech Co., Ltd.) having a longitudinal length of 3 cm and a horizontal length of 1 cm using a micropipette to prepare a sample No. 1. Further, the E. coli solution was dispensed onto only one surface of the filter paper. A sample No. 2 was prepared in the same manner.

Next, the sample No. 1 was immersed for 1 hour in a test tube containing 10 mL of a buffering solution (product name: Gibco PBS, produced by Thermo Fisher Scientific K.K.). Note that the time from the dispensing of the E. coli solution onto the filter paper to the immersion of the sample No. 1 in the buffering solution was set at 60 seconds to prevent the drying of the E. coli solution on the filter paper.

Then, 1 mL of the buffering solution (that will also be called a “1/1 solution”) after the immersion of the sample No. 1 was placed into a test tube containing 9 mL of a buffering solution to prepare a diluted solution (that will be called a “ 1/10 diluted solution”). Except for changing a dilution factor with a buffering solution, a 1/100 diluted solution, a 1/1000 diluted solution, and a 1/10000 diluted solution were prepared in the same manner.

Next, 0.050 mL was collected from the 1/1 solution and smeared on a stamp medium (Petan Check 25 PT1025, produced by EIKEN CHEMICAL CO., LTD.). This operation was repeatedly performed to generate two stamp media on which the 1/1 solution was smeared. The two stamp media were placed into a constant temperature bath (product name: IS600, manufactured by Yamato Scientific Co., Ltd.) and incubated for 24 hours at 37° C. The number of colonies generated on the two stamp media was counted, and the average of the number of the colonies was calculated.

For each of the 1/10 diluted solution, the 1/100 diluted solution, the 1/1000 diluted solution, and the 1/10000 diluted solution, two smeared stamp media were also generated in the same manner and incubated. Then, the number of colonies generated on each stamp medium corresponding to each diluted solution was counted, and the average of the number of the colonies was calculated. The results are shown in Table 1-1.

TABLE 1-1

| | | Sample No. 1 (blank) |
|---|---|---|
| 1/1 | Solution | >100 |
| 1/10 | Solution | >100 |
| 1/100 | Solution | 54 |
| 1/1000 | Solution | 4 |
| 1/10000 | Solution | 0 |

It is found from the results shown in Table 1-1 that the number of the colonies generated when the 1/100 diluted solution was incubated was 54, and that the number of the colonies present in 0.050 mL of the 1/1 solution corresponding to the sample No. 1 was therefore 54×10²=5400 (CFU).

Next, the following operation was conducted for the sample No. 2.

A concave part having a longitudinal length of 3.5 cm, a horizontal length of 1.5 cm, and a depth of 1.4 mm was provided at the center of a plastic flat plate having a longitudinal length of 30 cm, a horizontal length of 30 cm, and a depth of 5 mm. In the concave part, a filter paper having a longitudinal length of 3.5 cm and a horizontal length of 1.5 cm was laid. On the filter paper, the sample No. 2 was arranged with its E. coli solution dispensed surface opposed to the filter paper laid on the bottom of the concave part. Since the filter paper had a thickness of 0.2 mm, the depth of the concave part became 1 mm. Next, in the portion of the plastic flat plate where the filter paper was laid, the active oxygen supply device was arranged so that the longitudinal center of its opening aligned with the longitudinal center of the concave part, and so that the widthwise center of the opening aligned with the short-axis direction of the concave part. Accordingly, the distance between the opening part 106 of the active oxygen supply device and the surface of the filter paper on the side opposite to the opening became 1 mm. Next, the humidifying device was operated under the same conditions (with a relative humidity of 95% RH (and a temperature of 25° C.) at the exit of the emission unit), while applying an AC voltage with a sine waveform having an amplitude of 2.4 kVpp and a frequency of 80 kHz between both electrodes of the active oxygen supply device, thereby supplying an induced flow toward the filter paper. The supply time (treatment time) was set at 10 seconds.

Further, in a treatment processing using the active oxygen supply device, the time from the dispensing of the E. coli solution onto the filter paper to the immersion of the sample No. 2 in a buffering solution was set at 60 seconds to prevent the drying of the filter paper where the E. coli solution was dispensed as much as possible.

The sample No. 2 after the treatment was immersed for 1 hour in a test tube containing 10 mL of a buffering solution (product name: Gibco PBS, produced by Thermo Fisher Scientific K.K.) together with the filter paper laid on the bottom of the concave part. Next, 1 mL of the buffering solution (that will be called a “1/1 solution”) after the immersion was placed into a test tube containing 9 mL of a buffering solution to prepare a diluted solution ( 1/10 diluted solution). Except for changing a dilution factor with a buffering solution, a 1/100 diluted solution, a 1/1000 diluted solution, and a 1/10000 diluted solution were prepared in the same manner.

Next, 0.050 mL was collected from the 1/1 solution and smeared on a stamp medium (product name: Petan Check 25 PT1025, produced by EIKEN CHEMICAL CO., LTD.). This operation was repeatedly performed to generate two stamp media on which the 1/1 solution was smeared. The totally two stamp media were placed into a constant temperature bath (product name: IS600, manufactured by Yamato Scientific Co., Ltd.) and incubated for 24 hours at 37°° C. The number of colonies generated on each stamp medium corresponding to the 1/1 solution was counted, and the average of the number of the colonies was calculated. For each of the 1/10 diluted solution, the 1/100 diluted solution, the 1/1000 diluted solution, and the 1/10000 diluted solution, two smeared stamp media were also generated in the same manner and incubated. Then, the number of colonies generated on each stamp medium corresponding to each diluted solution was counted, and the average of the number of the colonies was calculated. The results are shown in Table 1-2.

TABLE 1-2

| | | Sample No. 2 |
|---|---|---|
| 1/1 | Solution | 36 |
| 1/10 | Solution | 3 |
| 1/100 | Solution | 0 |
| 1/1000 | Solution | 0 |
| 1/10000 | Solution | 0 |

As shown in Table 1-2, the number of colonies in 0.050 mL of the 1/1 solution corresponding to the sample No. 2 after the treatment was 36 (CFU), while the number of colonies in 0.050 mL of the 1/1 solution corresponding to the sample No. 1, which did not undergo the treatment with the active oxygen supply device, was 5400 (CFU). Thus, it is found that sterilization of 99.33% ((5400−36)/5400×100) was achieved by the treatment for 10 seconds with the active oxygen supply device according to this Example.

### Examples 2 and 3

The gas flow rate of the pump of a humidifying device in Example 1 was controlled so that the humidity measured at each opening part became 80% RH or 70% RH. Except for that, an active oxygen treatment device was manufactured in the same manner as Example 1. Using this active oxygen treatment device, an ozone generation amount obtained when only a plasma actuator was operated and an ozone generation amount obtained when both the plasma actuator and the humidifying device were operated were measured in the same manner as Example 1. Further, the active oxygen treatment device was subjected to a decolorization test for methylene blue and a sterilization test in the same manner as Example 1.

### Example 4

An active oxygen treatment device was manufactured with an emission unit to emit humidified air arranged upstream of an induced flow generated by a plasma actuator as shown in FIG. 1B. Specifically, the distance (denoted by symbol 108-2 in FIG. 1B) between the center of an emission unit 108 having a diameter Φ of 6 mm and an edge part 204 of a first electrode 203 of the plasma actuator was set at 10 mm. At the time, the distance between the center of the emission unit 108 and an air intake part 109 was 5 mm. Except for that, the active oxygen treatment device was manufactured in the same manner as the active oxygen treatment device according to Example 1.

Humidity at the air intake part 109 and humidity at an opening part 106 of the active oxygen treatment device were 90% RH and 88% RH, respectively. Using this active oxygen treatment device, an ozone generation amount obtained when only the plasma actuator was operated and an ozone generation amount obtained when both the plasma actuator and a humidifying device were operated were measured in the same manner as Example 1. Moreover, the active oxygen treatment device was subjected to a decolorization test for methylene blue and a sterilization test in the same manner as Example 1.

### Examples 5 and 6

The gas flow rate of the pump of a humidifying device in Example 4 was controlled so that humidity measured at an air intake part 109 became 76% RH or 66% RH. Except for that, an active oxygen treatment device was manufactured in the same manner as Example 4. Using this active oxygen treatment device, an ozone generation amount obtained when only a plasma actuator was operated and an ozone generation amount obtained when both the plasma actuator and the humidifying device were operated were measured in the same manner as Example 1. Further, the active oxygen treatment device was subjected to a decolorization test for methylene blue and a sterilization test in the same manner as Example 1.

### Example 7

An active oxygen supply device was manufactured in the same manner as Example 1 except that an applied voltage of a plasma actuator in Example 1 was changed from 2.4 kVpp to 2.0 kVpp. The active oxygen treatment device was then evaluated.

### Comparative Example 1

An active oxygen supply device was operated in a room where a relative humidity of 30% RH (and a temperature of 25° C.) were maintained, while a humidifying device of the active oxygen supply device was stopped in Example 1. As a result, humidity measured at an opening part 106 also became 30% RH. Using this active oxygen supply device, an ozone generation amount obtained when only a plasma actuator was operated and an ozone generation amount obtained when both the plasma actuator and the humidifying device were operated were measured in the same manner as Example 1. Further, the active oxygen supply device was subjected to a decolorization test for methylene blue and a sterilization test in the same manner as Example 1.

### Comparative Example 2

Except that a room had a relative humidity of 15% RH (and a temperature of 25° C.) in Comparative Example 1, an active oxygen supply device was evaluated in the same manner as Comparative Example 1.

The evaluation results of Examples 1 to 7 and Comparative Examples 1 and 2 are shown in Table 2.

TABLE 2

| | | | | Relative | | | | |
|---|---|---|---|---|---|---|---|---|
| | | Ozone | | humidity | | | | |
| | | concentration | | (% RH) | | | | |
| | | (μg/min) | | | Air | Reduction | Sterilization test | |
| | | Humidifying | Humidifying | Opening | intake | rate of | | Sterilization |
| | | device | device | part | part | absorbance | | rate |
| | | Off | On | 106 | 109 | (%) | (CFU) | (%) |
| Examples | 1 | 18 | 11 | 90 | 40 | 92 | 36 | 99.33% |
| | 2 | 18 | 12 | 80 | 39 | 91 | 55 | 98.98% |
| | 3 | 18 | 13 | 70 | 41 | 90 | 64 | 98.81% |
| | 4 | 18 | 11 | 88 | 90 | 88 | 83 | 98.46% |
| | 5 | 18 | 12 | 76 | 78 | 85 | 92 | 98.30% |
| | 6 | 18 | 13 | 66 | 69 | 83 | 112 | 97.93% |
| | 7 | 9 | 5 | 90 | 40 | 82 | 135 | 97.50% |
| Comparative | 1 | 18 | 18 | 30 | 30 | 24 | 4800 | 11.11% |
| Examples | 2 | 18 | 18 | 15 | 15 | 18 | 5100 | 5.56% |

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An active oxygen supply device comprising:
a humidifying device;
a housing having at least one opening part; and
a plasma actuator arranged inside of the housing, wherein
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the humidifying device humidifies the inside of the housing to generate active oxygen in the induced flow, and the induced flow results in an induced flow containing the active oxygen, and
the plasma actuator and the humidifying device are arranged so that the induced flow containing the active oxygen flows to an outside of the housing from the opening part.

2. The active oxygen supply device according to claim 1, wherein,
when a cross section of the plasma actuator in a thickness direction is viewed,
the first electrode and the second electrode are arranged diagonally opposite to each other via the dielectric in the thickness direction of the plasma actuator,
the first electrode is provided to coat a part of the first surface of the dielectric,
the first surface has an exposed part not coated with the first electrode,
at least a part of the exposed part and the second electrode overlap each other when the plasma actuator is viewed through from a side of the first electrode, and
the induced flow containing the ozone blows out along the exposed part of the dielectric overlapping the second electrode from an edge part of the first electrode on a side of the first direction in the cross section in the thickness direction.

3. The active oxygen supply device according to claim 1, wherein,
assuming that an edge part of the first electrode on the side of the first direction is an edge part A and an edge part of the second electrode on a side of a second direction opposite to the first direction is an edge part B in the cross section of the plasma actuator in the thickness direction,
the edge part B is positioned on the side of the second direction relative to the edge part A.

4. The active oxygen supply device according to claim 1, wherein,
assuming that an edge part of the first electrode on the side of the first direction is an edge part A and an edge part of the second electrode on the side of the second direction opposite to the first direction is an edge part B when the plasma actuator is viewed through from the side of the first electrode,
the edge part B is positioned on the side of the second direction relative to the edge part A.

5. The active oxygen supply device according to claim 1, wherein,
assuming that an edge part of the first electrode on the side of the first direction is an edge part A and an edge part of the second electrode on a side of a second direction opposite to the first direction is an edge part B in the cross section of the plasma actuator in the thickness direction,
the edge part B is positioned on the side of the first direction relative to the edge part A.

6. The active oxygen supply device according to claim 1, wherein,
assuming that an edge part of the first electrode on the side of the first direction is an edge part A and an edge part of the second electrode on a side of a second direction opposite to the first direction is an edge part B when the plasma actuator is viewed through from the side of the first electrode,
the edge part B is positioned on the side of the first direction relative to the edge part A.

7. The active oxygen supply device according to claim 1, wherein,
assuming that an edge part of the first electrode on the side of the first direction is an edge part A and an edge part of the second electrode on a side of a second direction opposite to the first direction is an edge part B when the plasma actuator is viewed through from the side of the first electrode,
the edge part A and the edge part B align with each other in a thickness direction of the dielectric.

8. The active oxygen supply device according to claim 1, wherein,
an ozone generation amount per unit time in a state in which the induced flow is not humidified is at least 8 μg/min in the plasma actuator.

9. The active oxygen supply device according to claim 1, wherein
the first surface of the dielectric has an exposed part not coated with the first electrode, and
a narrow angle θ formed between an extension line in a direction along the exposed part of the first surface of the dielectric from an edge part of the first electrode of the plasma actuator and a horizontal plane is 0° to 90° when the opening part of the active oxygen supply device is directed vertically downward.

10. The active oxygen supply device according to claim 1, wherein
a distance between the humidifying device and the plasma actuator is not more than 10 mm.

11. The active oxygen supply device according to claim 1, wherein
the humidifying device is arranged to be capable of humidifying an object to be treated, which is positioned outside of the housing, via the opening part.

12. The active oxygen supply device according to claim 1, wherein
the plasma actuator, the humidifying device, and the opening part are arranged in this order toward a longitudinal direction inside of the housing.

13. A treatment device treating a surface of an object to be treated using active oxygen, the treatment device comprising:
a humidifying device;
a housing having at least one opening part; and
a plasma actuator arranged inside of the housing, wherein
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the humidifying device humidifies the induced flow containing the ozone to generate active oxygen in the induced flow, and the induced flow results in an induced flow containing the active oxygen, and
the plasma actuator and the humidifying device are arranged so that the induced flow containing the active oxygen flows to an outside of the housing from the opening part.

14. The treatment device using active oxygen according to claim 13, wherein
the humidifying device is arranged to be capable of humidifying the surface of the object to be treated.

15. A treatment method for treating a surface of an object to be treated using active oxygen, the treatment method comprising:
a step of preparing a treatment device using active oxygen, wherein
the treatment device using the active oxygen comprises
a humidifying device,
a housing having at least one opening part, and
a plasma actuator arranged inside of the housing,
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the humidifying device humidifies the induced flow containing the ozone to generate active oxygen in the induced flow, and the induced flow results in an induced flow containing the active oxygen, and
the plasma actuator and the humidifying device are arranged so that the induced flow containing the active oxygen flows to an outside of the housing from the opening part,
the treatment method further comprising
a step of arranging the prepared treatment device using the active oxygen and the object to be treated at relative positions so that the surface of the object to be treated is exposed when the induced flow containing the active oxygen flows out from the opening part, and
a step of causing the induced flow to flow out from the opening part to treat the surface of the object using the active oxygen.

16. The treatment method using active oxygen according to claim 15, wherein
a distance between the humidifying device and the surface of the object to be treated is not more than 10 mm.
